(19) United States
(12) Patent Application Publication
(10) Pub. No.: US 2010/0234464 A1
(43) Pub. Date: Sep. 16, 2010

(54) COMPOUNDS FOR THE TREATMENT OF METABOLIC DISORDERS

(75) Inventors: Shalini Sharma, Gaithersburg, MD (US); Reid W. von Borstel, Potomac, MD (US)

Correspondence Address: Wellstat Management Company. LLC, LEGAL DEPARTMENT, 930 CLOPPER ROAD, GAITHERSBURG, MD, 20878, US

(73) Assignee: WELLSTAT THERAPEUTICS CORPORATION, Gaithersburg, MD (US)

(21) Appl. No.: 12/304,007

(22) PCT Filed: Jun. 8, 2007
(86) PCT No.: PCT/US07/70691

§ 371 Date: Dec. 9, 2008

Related U.S. Application Data
(60) Provisional application No. 60/804,336, filed on Jun. 9, 2006.

(51) Int. Cl. A61K 31/192; A61P 3/00; A61P 3/10; A61P 3/04; A61P 9/10; C07C 59/135

(52) U.S. Cl. 514/570; 562/472

(57) ABSTRACT

3-(2,4-Bis(trifluoromethyl)benzyloxy)phenylacetic acid, 4-(2,6-Dimethylbenzyloxy)-phenylacetic acid, and their pharmaceutically acceptable salts are useful for the treatment of various metabolic disorders, such as insulin resistance syndrome, diabetes, polycystic ovary syndrome, hyperlipidemia, fatty liver disease, cachexia, obesity, atherosclerosis and arteriosclerosis.

### BACKGROUND OF THE INVENTION

Diabetes mellitus is a major cause of morbidity and mortality. Chronically elevated blood glucose leads to debilitating complications: nephropathy, often necessitating dialysis or renal transplant; peripheral neuropathy; retinopathy leading to blindness; ulceration of the legs and feet, leading to amputation; fatty liver disease, sometimes progressing to cirrhosis; and vulnerability to coronary artery disease and myocardial infarction.

There are two primary types of diabetes. Type I, or insulin-dependent diabetes mellitus (IDDM) is due to autoimmune destruction of insulin-producing beta cells in the pancreatic islets. The onset of this disease is usually in childhood or adolescence. Treatment consists primarily of multiple daily injections of insulin, combined with frequent testing of blood glucose levels to guide adjustment of insulin doses, because excess insulin can cause hypoglycemia and consequent impairment of brain and other functions.

Type II, or noninsulin-dependent diabetes mellitus (NIDDM) typically develops in adulthood. NIDDM is associated with resistance of glucose-utilizing tissues like adipose tissue, muscle, and liver, to the actions of insulin. Initially, the pancreatic islet beta cells compensate by secreting excess insulin. Eventual islet failure results in decompensation and chronic hyperglycemia. Conversely, moderate islet insufficiency can precede or coincide with peripheral insulin resistance. There are several classes of drugs that are useful for treatment of NIDDM: 1) insulin releasers, which directly stimulate insulin release, carrying the risk of hypoglycemia; 2) prandial insulin releasers, which potentiate glucose-induced insulin secretion, and must be taken before each meal; 3) biguanides, including metformin, which attenuate hepatic gluconeogenesis (which is paradoxically elevated in diabetes); 4) insulin sensitizers, for example the thiazolidinedione derivatives rosiglitazone and pioglitazone, which improve peripheral responsiveness to insulin, but which have side effects like weight gain, edema, and occasional liver toxicity; 5) insulin injections, which are often necessary in the later stages of NIDDM when the islets have failed under chronic hyperstimulation.

Insulin resistance can also occur without marked hyperglycemia, and is generally associated with atherosclerosis, obesity, hyperlipidemia, and essential hypertension. This cluster of abnormalities constitutes the “metabolic syndrome” or “insulin resistance syndrome”. Insulin resistance is also associated with fatty liver, which can progress to chronic inflammation (NASH; “nonalcoholic steatohepatitis”), fibrosis, and cirrhosis. Cumulatively, insulin resistance syndromes, including but not limited to diabetes, underlie many of the major causes of morbidity and death of people over age 40.

Despite the existence of such drugs, diabetes remains a major and growing public health problem. Late stage complications of diabetes consume a large proportion of national health care resources. There is a need for new orally active therapeutic agents which effectively address the primary defects of insulin resistance and islet failure with fewer or milder side effects than existing drugs.

Currently there are no safe and effective treatments for fatty liver disease. Therefore such a treatment would be of value in treating this condition.

WO 04/073611 (Wellstat Therapeutics Corp.) describes a genus of compounds that generically encompasses the compounds of this invention.

### SUMMARY OF THE INVENTION

This invention provides a biologically active agent as described below. This invention provides the use of the biologically active agent described below in the manufacture of a medicament for the treatment of insulin resistance syndrome, diabetes, cachexia, hyperlipidemia, fatty liver disease, obesity, atherosclerosis or arteriosclerosis. This invention provides methods of treating a mammalian subject with insulin resistance syndrome, diabetes, cachexia, hyperlipidemia, fatty liver disease, obesity, atherosclerosis or arteriosclerosis comprising administering to the subject an effective amount of the biologically active agent described below. This invention provides a pharmaceutical composition comprising the biologically active agent described below and a pharmaceutically acceptable carrier.

The biologically active agent of this invention that has been tested showed activity in the biological activity assays described below, which are established animal models of human diabetes and insulin resistance syndrome. It is believed that all biologically active agents of this invention will also have activity in one or more of these assays. Therefore such agents would be useful in the treatment of diabetes and insulin resistance syndrome.

### DETAILED DESCRIPTION OF THE INVENTION

As used herein the transitional term “comprising” is open-ended. A claim utilizing this term can contain elements in addition to those recited in such claim.

### Compounds of the Invention

The biologically active agent in accordance with this invention is selected from the group consisting of: 3-(2,4-Bis(trifluoromethyl)benzyloxy)phenylacetic acid; 4-(2,6-Dimethylbenzyloxy)phenylacetic acid; and their pharmaceutically acceptable salts.

Certain chemical compounds are referred to herein by their chemical name or by the two-letter code shown below. Compounds DN and DO are included within the scope of the biologically active agents of this invention.
BI 4-(3-(2,6-Dimethylbenzyloxy)phenyl)-4-oxobutyric acid
CF 3-(2,6-Dimethylbenzyloxy)phenylacetic acid
DN 3-(2,4-Bis(trifluoromethyl)benzyloxy)phenylacetic acid
DO 4-(2,6-Dimethylbenzyloxy)phenylacetic acid

In a preferred embodiment of the biologically active agent of this invention, the agent is in substantially (at least 98%) pure form.

The biologically active agents of the present invention can be made as described in WO 04/073611, and as described in the examples below. The contents of WO 04/073611 are incorporated herein by reference.

### Use in Methods of Treatment

This invention provides a method for treating a mammalian subject with a condition selected from the group consisting of insulin resistance syndrome, diabetes (both primary essential diabetes such as Type I Diabetes or Type II Diabetes and secondary nonessential diabetes) and polycystic ovary syndrome, comprising administering to the subject an amount of a biologically active agent as described herein effective to treat the condition. In accordance with the method of this invention a symptom of diabetes or the chance of developing a symptom of diabetes, such as atherosclerosis, obesity, hypertension, hyperlipidemia, fatty liver disease, nephropathy, neuropathy, retinopathy, foot ulceration and cataracts, each such symptom being associated with diabetes, can be reduced. This invention also provides a method for treating hyperlipidemia comprising administering to the subject an amount of a biologically active agent as described herein effective to treat the condition. Compounds reduce serum triglycerides and free fatty acids in hyperlipidemic animals. This invention also provides a method for treating cachexia comprising administering to the subject an amount of a biologically active agent as described herein effective to treat the cachexia. This invention also provides a method for treating obesity comprising administering to the subject an amount of a biologically active agent as described herein effective to treat the condition. This invention also provides a method for treating a condition selected from atherosclerosis or arteriosclerosis comprising administering to the subject an amount of a biologically active agent as described herein effective to treat the condition. The active agents of this invention are effective to treat hyperlipidemia, fatty liver disease, cachexia, obesity, atherosclerosis or arteriosclerosis whether or not the subject has diabetes or insulin resistance syndrome. The agent can be administered by any conventional route of systemic administration. Preferably the agent is administered orally. Accordingly, it is preferred for the medicament to be formulated for oral administration. Other routes of administration that can be used in accordance with this invention include rectally, parenterally, by injection (e.g. intravenous, subcutaneous, intramuscular or intraperitioneal injection), or nasally.

Further embodiments of each of the uses and methods of treatment of this invention comprise administering any one of the embodiments of the biologically active agents described above. In the interest of avoiding unnecessary redundancy, each such agent and group of agents is not being repeated, but they are incorporated into this description of uses and methods of treatment as if they were repeated.

Many of the diseases or disorders that are addressed by the compounds of the invention fall into two broad categories: Insulin resistance syndromes and consequences of chronic hyperglycemia. Dysregulation of fuel metabolism, especially insulin resistance, which can occur in the absence of diabetes (persistent hyperglycemia) per se, is associated with a variety of symptoms, including hyperlipidemia, atherosclerosis, obesity, essential hypertension, fatty liver disease (NASH; nonalcoholic steatohepatitis), and, especially in the context of cancer or systemic inflammatory disease, cachexia. Cachexia can also occur in the context of Type I Diabetes or late-stage Type II Diabetes. By improving tissue fuel metabolism, active agents of the invention are useful for preventing or amelioriating diseases and symptoms associated with insulin resistance. While a cluster of signs and symptoms associated with insulin resistance may coexist in an individual patient, it many cases only one symptom may dominate, due to individual differences in vulnerability of the many physiological systems affected by insulin resistance. Nonetheless, since insulin resistance is a major contributor to many disease conditions, drugs which address this cellular and molecular defect are useful for prevention or amelioration of virtually any symptom in any organ system that may be due to, or exacerbated by, insulin resistance.

When insulin resistance and concurrent inadequate insulin production by pancreatic islets are sufficiently severe, chronic hyperglycemia occurs, defining the onset of Type II diabetes mellitus (NIDDM). In addition to the metabolic disorders related to insulin resistance indicated above, disease symptoms secondary to hyperglycemia also occur in patients with NIDDM. These include nephropathy, peripheral neuropathy, retinopathy, microvascular disease, ulceration of the extremities, and consequences of nonenzymatic glycosylation of proteins, e.g. damage to collagen and other connective tissues. Attenuation of hyperglycemia reduces the rate of onset and severity of these consequences of diabetes. Because active agents and compositions of the invention help to reduce hyperglycemia in diabetes, they are useful for prevention and amelioration of complications of chronic hyperglycemia.

Both human and non-human mammalian subjects can be treated in accordance with the treatment method of this invention. The optimal dose of a particular active agent of the invention for a particular subject can be determined in the clinical setting by a skilled clinician. In the case of oral administration to a human for treatment of disorders related to insulin resistance, diabetes, hyperlipidemia, fatty liver disease, cachexia or obesity the agent is generally administered in a daily dose of from 1 mg to 400 mg, administered once or twice per day. In the case of oral administration to a mouse the agent is generally administered in a daily dose from 1 to 300 mg of the agent per kilogram of body weight. Active agents of the invention are used as monotherapy in diabetes or insulin resistance syndrome, or in combination with one or more other drugs with utility in these types of diseases, e.g. insulin releasing agents, prandial insulin releasers, biguanides, or insulin itself. Such additional drugs are administered in accord with standard clinical practice. In some cases, agents of the invention will improve the efficacy of other classes of drugs, permitting lower (and therefore less toxic) doses of such agents to be administered to patients with satisfactory therapeutic results. Established safe and effective dose ranges in humans for representative compounds are: metformin 500 to 2550 mg/day; glyburide 1.25 to 20 mg/day; GLUCOVANCE (combined formulation of metformin and glyburide) 1.25 to 20 mg/day glyburide and 250 to 2000 mg/day metformin; atorvastatin 10 to 80 mg/day; lovastatin 10 to 80 mg/day; pravastatin 10 to 40 mg/day; and simvastatin 5-80 mg/day; clofibrate 2000 mg/day; gemfibrozil 1200 to 2400 mg/day, rosiglitazone 4 to 8 mg/day; pioglitazone 15 to 45 mg/day; acarbose 75-300 mg/day; repaglinide 0.5 to 16 mg/day.

Type I Diabetes Mellitus: A patient with Type I diabetes manages their disease primarily by self-administration of one to several doses of insulin per day, with frequent monitoring blood glucose to permit appropriate adjustment of the dose and timing of insulin administration. Chronic hyperglycemia leads to complications such as nephropathy, neuropathy, retinopathy, foot ulceration, and early mortality; hypoglycemia due to excessive insulin dosing can cause cognitive dysfunction or unconsciousness. A patient with Type I diabetes is treated with 1 to 400 mg/day of an active agent of this invention, in tablet or capsule form either as a single or a divided dose. The anticipated effect will be a reduction in the dose or frequency of administration of insulin required to maintain blood glucose in a satisfactory range, and a reduced incidence and severity of hypoglycemic episodes. Clinical outcome is monitored by measurement of blood glucose and glycosylated hemoglobin (an index of adequacy of glycemic control integrated over a period of several months), as well as by reduced incidence and severity of typical complications of diabetes. A biologically active agent of this invention can be administered in conjunction with islet transplantation to help maintain the anti-diabetic efficacy of the islet transplant.

Type II Diabetes Mellitus: A typical patient with Type II diabetes (NIDDM) manages their disease by programs of diet and exercise as well as by taking medications such as metformin, glyburide, repaglinide, rosiglitazone, or acarbose, all of which provide some improvement in glycemic control in some patients, but none of which are free of side effects or eventual treatment failure due to disease progression. Islet failure occurs over time in patients with NIDDM, necessitating insulin injections in a large fraction of patients. It is anticipated that daily treatment with an active agent of the invention (with or without additional classes of antidiabetic medication) will improve glycemic control, reduce the rate of islet failure, and reduce the incidence and severity of typical symptoms of diabetes. In addition, active agents of the invention will reduce elevated serum triglycerides and fatty acids, thereby reducing the risk of cardiovascular disease, a major cause of death of diabetic patients. As is the case for all other therapeutic agents for diabetes, dose optimization is done in individual patients according to need, clinical effect, and susceptibility to side effects.

Hyperlipidemia: Elevated triglyceride and free fatty acid levels in blood affect a substantial fraction of the population and are an important risk factor for atherosclerosis and myocardial infarction. Active agents of the invention are useful for reducing circulating triglycerides and free fatty acids in hyperlipidemic patients. Hyperlipidemic patients often also have elevated blood cholesterol levels, which also increase the risk of cardiovascular disease. Cholesterol-lowering drugs such as HMG-CoA reductase inhibitors (“statins”) can be administered to hyperlipidemic patients in addition to agents of the invention, optionally incorporated into the same pharmaceutical composition.

Fatty Liver Disease: A substantial fraction of the population is affected by fatty liver disease, also known as nonalcoholic steatohepatitis (NASH); NASH is often associated with obesity and diabetes. Hepatic steatosis, the presence of droplets of triglycerides with hepatocytes, predisposes the liver to chronic inflammation (detected in biopsy samples as infiltration of inflammatory leukocytes), which can lead to fibrosis and cirrhosis. Fatty liver disease is generally detected by observation of elevated serum levels of liver-specific enzymes such as the transaminases ALT and AST, which serve as indices of hepatocyte injury, as well as by presentation of symptoms which include fatigue and pain in the region of the liver, though definitive diagnosis often requires a biopsy. The anticipated benefit is a reduction in liver inflammation and fat content, resulting in attenuation, halting, or reversal of the progression of NASH toward fibrosis and cirrhosis.

### Pharmaceutical Compositions

This invention provides a pharmaceutical composition comprising a biologically active agent as described herein and a pharmaceutically acceptable carrier. Further embodiments of the pharmaceutical composition of this invention comprise any one of the embodiments of the biologically active agents described above. In the interest of avoiding unnecessary redundancy, each such agent and group of agents is not being repeated, but they are incorporated into this description of pharmaceutical compositions as if they were repeated.

Preferably the composition is adapted for oral administration, e.g. in the form of a tablet, coated tablet, dragee, hard or soft gelatin capsule, solution, emulsion or suspension. In general the oral composition will comprise from 1 mg to 400 mg of such agent. It is convenient for the subject to swallow one or two tablets, coated tablets, dragees, or gelatin capsules per day. However the composition can also be adapted for administration by any other conventional means of systemic administration including rectally, e.g. in the form of suppositories, parenterally, e.g. in the form of injection solutions, or nasally.

The biologically active compounds can be processed with pharmaceutically inert, inorganic or organic carriers for the production of pharmaceutical compositions. Lactose, corn starch or derivatives thereof, talc, stearic acid or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active ingredient no carriers are, however, usually required in the case of soft gelatin capsules, other than the soft gelatin itself. Suitable carriers for the production of solutions and syrups are, for example, water, polyols, glycerol, vegetable oils and the like. Suitable carriers for suppositories are, for example, natural or hardened oils, waxes, fats, semil-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, coating agents or antioxidants. They can also contain still other therapeutically valuable substances, particularly antidiabetic or hypolipidemic agents that act through mechanisms other than those underlying the effects of the compounds of the invention. Agents which can advantageously be combined with compounds of the invention in a single formulation include but are not limited to biguanides such as metformin, insulin releasing agents such as the sulfonylurea insulin releaser glyburide and other sulfonylurea insulin releasers, cholesterol-lowering drugs such as the “statin” HMG-CoA reductase inhibitors such as atrovastatin, lovastatin, pravastatin and simvastatin, PPAR-alpha agonists such as clofibrate and gemfibrozil, PPAR-gamma agonists such as thiazolidinediones (e.g. rosiglitazone and pioglitazone, alpha-glucosidase inhibitors such as acarbose (which inhibit starch digestion), and prandial insulin releasers such as repaglinide. The amounts of complementary agents combined with compounds of the invention in single formulations are in accord with the doses used in standard clinical practice. Established safe and effective dose ranges for certain representative compounds are set forth above.

The invention will be better understood by reference to the following examples which illustrate but do not limit the invention described herein.

### EXAMPLES

### Example 1

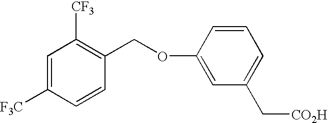

### 3-(2,4-Bis(trifluoromethyl)benzyloxy)phenylacetic acid

### Step A: Preparation of Ethyl 3-hydroxyphenylacetate

A solution of 3-Hydroxyphenylacetic acid (25 g, 164.31 mmol) and p-Toluenesulfonic acid monohydrate (3.49 g, 18.3 mmol) in abs ethanol (250 ml) was refluxed for 4 hours or until all the starting material is consumed. The reaction mixture was concentrated, diluted with ethyl acetate and washed with water. The organic layer was dried over Na₂SO₄, filtered, concentrated, and purified by flash chromatography on a silica gel column (hex: ethyl acetate 2:1) to give the title compound.

¹H NMR (270 MHz, CDCl₃): 1.2 (t, 3H); 3.5 (s, 2H); 4.1 (q, 2H); 6.6-7.2 (m, 4H).

### Step B: Preparation of Ethyl 3-(2,4-bis(trifluoromethyl)benzyloxy)phenylacetate

To a solution of Ethyl 3-hydroxyphenylacetate (3.22 g, 17.9 mmol) and potassium carbonate (2.92 g, 21.2 mmol) in dry N,N-Dimethylformamide (15 ml) was added 2,4-Bis(trifluoromethyl)benzyl bromide (5 g, 16.3 mmol). The reaction mixture was stirred at room temperature for 5 hours, quenched with water and concentrated in vaccuo. The crude residue was taken in ethyl acetate and washed with water and brine. The aqueous layer was extracted twice with ethyl acetate. The combined organic layers were dried over Na₂SO₄, filtered, concentrated and purified by flash chromatography on a silica gel column (hex: ether, 5:1) to give the title compound.

¹H NMR (270 MHz, CDCl₃): 1.2 (t, 3H); 3.6 (s, 2H); 4.1 (q, 2H); 5.3 (s, 2H); 6.8-7.0 (m, 3H); 7.3 (m, 1H); 7.8 (d, 1H); 7.9-8.0 (m, 2H).

### Step C: Preparation of 3-(2,4-Bis(trifluoromethyl)benzyloxy)phenylacetic acid

To a stirred solution of Ethyl 3-(2,4-bis(trifluoromethyl)benzyloxy)phenylacetate (Step B, 3.88 g, 9.5 mmol) in absolute ethanol (50 ml) was added 1N NaOH (20 ml) at room temperature. The reaction mixture was stirred for 3 hours, acidified by 1N HCl to pH 3-4, and concentrated. The residue was taken into chloroform and washed with 0.1N HCl, dried over Na₂SO₄, filtered, concentrated and purified by flash chromatography on a silica gel column (chloroform:methanol (95:5) spiked with acetic acid) to give the title compound.

¹H NMR (270 MHz, CDCl₃): 3.7 (s, 2H); 5.3 (s, 2H); 6.8-7.0 (m, 3H); 7.3 (m, 1H); 7.8 (d, 1H); 7.9-8.0 (m, 2H).

### Example 2

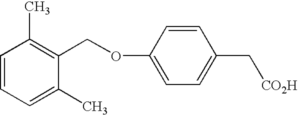

### 4-(2,6-Dimethylbenzyloxy)phenylacetic acid

### Step A: Preparation of Ethyl 4-hydroxyphenylacetate

A solution of 4-Hydroxyphenylacetic acid (25 g, 164.31 mmol) and p-Toluenesulfonic acid monohydrate (3.49 g, 18.3 mmol) in abs ethanol (250 ml) was refluxed for 4 hours or until all the starting material is consumed. The reaction mixture was concentrated, diluted with ethyl acetate and washed with water. The organic layer was dried over Na₂SO₄, filtered, concentrated, and purified by flash chromatography on a silica gel column (hex: ethyl acetate 2:1) to give the title compound.

¹H NMR (270 MHz, CDCl₃): 1.2 (t, 3H); 3.6 (s, 2H); 4.1 (q, 2H); 7.0 (d, 2H); 7.1 (d, 2H).

### Step B: Preparation of Ethyl 4-(2,6-dimethylbenzyloxy)phenylacetate

A solution of 2,6-Dimethylbenzyl alcohol (3 g, 22.0 mmol) and diisopropyl azodicarboxylate (DIAD, 4.86 g, 24 mmol) in THF (20 ml) was added drop wise to a solution of Ethyl 4-hydroxyphenylacetate (Step A, 4.36 g, 24.2 mmol) and triphenylphosphine (6.30 g, 24 mmol) in THF (100 ml) at 0° C. The reaction mixture was stirred at the same temperature for 4 hours, diluted with ether and washed with water. The organic layer was dried over Na₂SO₄, filtered, concentrated, and purified by flash chromatography on a silica gel column (hex: ethyl acetate 4:1) to give the title compound.

¹H NMR (270 MHz, CDCl₃): 1.2 (t, 3H); 2.4 (s, 6H); 3.6 (s, 2H); 4.1 (q, 2H); 5.0 (s, 2H); 7.0 (d, 2H); 7.1 (d, 2H); 7.2-7.3 (m, 3H).

### Step C: Preparation of 4-(2,6-Dimethylbenzyloxy)phenylacetic acid

To a stirred solution of Ethyl 4-(2,6-dimethylbenzyloxy)phenylacetate (Step B, 6.67 g, 22.4 mmol) in absolute ethanol (100 ml) was added 1N NaOH (50 ml) at room temperature. The reaction mixture was stirred for 3 hours, acidified by 1N HCl to pH 3-4, and concentrated. The residue was taken into chloroform and washed with 0.1N HCl, dried over Na₂SO₄, filtered, concentrated and purified by flash chromatography on a silica gel column (chloroform:methanol (95:5) spiked with acetic acid) to give the title compound.

¹H NMR (270 MHz, CDCl₃): 2.4 (s, 6H); 3.6 (s, 2H); 5.0 (s, 2H); 7.0 (d, 2H); 7.1 (d, 2H); 7.2-7.3 (m, 3H).

### Example 3

### Antidiabetic Efficacy of Oral Compound DN in Male Db/Db Mice

Male db/db mice have a defect in the receptor for the appetite-regulating protein leptin and consequently develop hyperphagia, obesity, insulin resistance, hyperglycemia, and hypertriglyceridemia. Furthermore, male db/db mice undergo progressive islet failure, resulting in a transition from hyperinsulinemia to insulin deficiency.

Male db/db mice (10 weeks old) were divided into weight-matched groups of 5 mice each. Age-matched male C57BL/6 mice were used as lean control animals. Mice received once-daily oral (gavage) doses of the vehicle (1% hydroxypropylmethylcellulose in water), Compound BI, Compound CF, or Compound DN for two weeks. Treatment groups and drug doses were as follows.
1. Vehicle
2. Compound BI 100 mg/kg/day
3. Compound CF 100 mg/kg/day
4. Compound DN 100 mg/kg/day

Nonfasting blood samples were collected after 14 days of treatment for determination of serum glucose, triglycerides, and free fatty acids.

After treatment for two weeks, vehicle-treated mice were severely hyperglycemic and also had elevated levels of serum triglyceride.

As shown in Tables 1 and 2, administration of either Compound BI, Compound CF or Compound DN significantly reduced nonfasting serum glucose and triglycerides relative to values observed in vehicle-treated mice.

TABLE 1
Serum glucose in db/db mice treated with Compound BI,
Compound CF or Compound DN for 2 weeks

| | Glucose (mg/dL) |
|---|---|
| N = 5 mice/group | Mean ± SD |
| Vehicle | 735.0 ± 66.0 |
| Compound BI | 257.0 ± 57.0* |
| Compound CF | 441.0 ± 162.0* |
| Compound DN | 396.0 ± 84.0* |

*p < 0.05 compared to Vehicle (Student's T-test)

TABLE 2
Serum triglycerides in db/db mice treated for 2 weeks

| | Triglycerides |
|---|---|
| | (mg/dL) |
| N = 5 mice/group | Mean ± SEM |
| Vehicle | 220.8 ± 62.1 |
| Compound BI | 85.8 ± 13.0* |
| Compound CF | 90.5 ± 17.4* |
| Compound DN | 157.2 ± 40.3 |

*p < 0.05 lower than Vehicle group (Student's T-test)

1. (canceled)

2. (canceled)

3. A method for treating a mammalian subject with a condition selected from the group consisting of insulin resistance syndrome, diabetes, polycystic ovary syndrome, hyperlipidemia, fatty liver disease, cachexia, obesity, atherosclerosis and arteriosclerosis comprising administering to the subject an amount of a biologically active agent,
wherein the agent is selected from the group consisting of:
3-(2,4-Bis(trifluoromethyl)benzyloxy)phenylacetic acid;
4-(2,6-Dimethylbenzyloxy)phenylacetic acid;
and their pharmaceutically acceptable salts.

4. The method of claim 3, wherein the subject is a human.

5. The method of claim 4, wherein the agent is administered orally in an amount from one milligram to four hundred milligrams per day.

6. The method of claim 3, wherein the condition is insulin resistance syndrome or Type II Diabetes.

7. The method of claim 3, wherein the treatment reduces a symptom of diabetes or the chances of developing a symptom of diabetes, wherein the symptom is selected from the group consisting of: atherosclerosis, obesity, hypertension, hyperlipidemia, fatty liver disease, nephropathy, neuropathy, retinopathy, foot ulceration and cataracts, associated with diabetes.

8. A pharmaceutical composition, comprising a pharmaceutically acceptable carrier and from one milligram to four hundred milligrams of a biologically active agent,
wherein the agent is selected from the group consisting of:
3-(2,4-Bis(trifluoromethyl)benzyloxy)phenylacetic acid;
4-(2,6-Dimethylbenzyloxy)phenylacetic acid;
and their pharmaceutically acceptable salts.

9. The pharmaceutical composition of claim 8 in oral dosage form.

10. A compound, 3-(2,4-Bis(trifluoromethyl)benzyloxy)phenylacetic acid, or a pharmaceutically acceptable salt thereof.

11. A compound, 4-(2,6-Dimethylbenzyloxy)phenylacetic acid, or a pharmaceutically acceptable salt thereof.
